(19) United States
(12) Patent Application Publication
(10) Pub. No.: US 2011/0200706 A1
(43) Pub. Date: Aug. 18, 2011

(54) METHOD FOR PRODUCING COOKED RICE FOOD, AND ENZYME PREPARATION FOR IMPROVING COOKED RICE FOOD

(75) Inventors: Noriaki YAMADA, Kawasaki-shi (JP); Hiroaki SATO, Kawasaki-shi (JP); Tomohiro KODERA, Kawasaki-shi (JP)

(73) Assignee: AJINOMOTO CO., INC., Chuo-ku (JP)

(21) Appl. No.: 13/035,128

(22) Filed: Feb. 25, 2011

Related U.S. Application Data
(63) Continuation of application No. PCT/JP09/66875, filed on Sep. 18, 2009.

(30) Foreign Application Priority Data
Sep. 25, 2008 (JP) 2008-246481

(51) Int. Cl. A23L 1/105; A23L 1/182; C12N 9/10

(52) U.S. Cl. 426/10; 426/618; 435/193

(57) ABSTRACT

A cooked rice food having improved physical properties and eating quality and an enzyme preparation for a cooked rice food can be produced by using transglutaminase and/or α-glucosidase and glucose oxidase.

### CROSS REFERENCES TO RELATED APPLICATIONS

This application is a continuation of International Patent Application No. PCT/JP2009/066875, filed on Sep. 18, 2009, and claims priority to Japanese Patent Application No. 246481/2008, filed on Sep. 25, 2008, both of which are incorporated herein by reference in their entireties.

### BACKGROUND OF THE INVENTION

1. Field of the Invention

The present invention relates to methods for producing a cooked rice food and an enzyme preparations for improving a cooked rice food, in which transglutaminase and/or α-glucosidase are used together with glucose oxidase.

2. Discussion of the Background

Many foods are composed of various components such as starch, proteins, saccharides and lipids which combinedly constitute the textures of the foods. Among all, starch and proteins largely contribute to food texture and changes of the starch with the passage of time are considered as particularly important.

When allowed to stand at an ordinary or low temperature, gelatinized starch liberates water and thus hardens. A number of studies have been made on this phenomenon that is called retrogradation of starch. To prevent retrogradation, it is generally required to maintain starch at a temperature of 80° C. or higher, quickly dry starch to reduce the water content thereof to 15% or less, maintain starch in an alkaline state of pH 13 or higher, etc. Commonly known methods for preventing retrogradation comprise adding a saccharide (glucose, fructose, liquid sugar, etc.), soybean protein, wheat gluten, a fatty acid ester or a polysaccharide (yam, konjac, etc.) to starch-containing foods. For example, JP-A-59-2664 discloses a method which comprises adding a thickener, a surfactant, etc. However, these methods cause significant taste changes and can exert only unstable effects and, therefore, the aforesaid problem cannot be sufficiently solved thereby.

As a means for preventing retrogradation, it is also known to add enzymes. For example, JP-A-58-86050 discloses a method for improving cooked rice by adding an enzyme such as amylase, protease or lipase to polished rice, further adding sodium chloride and cyclodextrin thereto and then cooking the obtained mixture. JP-A-60-199355 discloses a method for preventing retrogradation of cooked rice by spraying cooked rice with an aqueous solution of saccharifying amylase (β-amylase, glucoamylase). Although attempts have been made to improve the qualities of cooked rice by adding various enzyme preparations to rice as discussed above, no remarkable effect has not been established so far.

According to WO2005/096839, soft and sticky cooked rice that is hardly degraded with the passage of time can be obtained by adding, as a quality-improving agent for starch-containing foods, transglucosidase being α-glucosidase to rice in the cooking step. Although this agent is effective to a certain degree, there still remain some points to be improved, i.e., loosening rice grains sticking together and improving the properties. Thus, it is very difficult by any of these methods to achieve both improvements in the texture of cooked rice at the point of the finish of cooking and maintenance of the thus improved texture for a long period of time.

Relating to the application of transglutaminase to cooked rice, there has been disclosed a method for producing cooked rice, which suffers from no degradation in flavor even in the case of storing for a long time after cooking and to which stickiness is particularly added, by adding transglutaminase, a partially hydrolyzed protein, an oligosaccharide and a sugar alcohol to rice cooking water and then cooking. According to this disclosure, although the cooked rice prepared by adding transglutaminase alone has preferred crispness, it is inferior in stickiness and swallowing feeling to the cooked rice prepared by adding transglutaminase together with partially hydrolyzed wheat protein (JP-A-9-206006). Moreover, Japanese Patent No. 3310081 discloses a method for preventing degradation of starch in cooked rice with increased water content, during storage or distribution at a low temperature, by adding transglutaminase. However, the texture of rice grains in such cooked rice with increased water content is largely different from the texture of common cooked rice.

Since cooked rice contains a large amount of glucose, glucose oxidase likely exerts some effect thereon. However, it has never been reported to use this enzyme for improving the properties of cooked rice. Japanese Patent No. 3718495 discloses a method for producing a diet food comprising cooked rice by reducing the glucose content of cooked rice with the use of glucose oxidase. Japanese Patent No. 2808060 discloses a method for preventing degradation, that is caused by the oxidation of lipids by dissolved oxygen incorporated into glucose, by adding glucose oxidase and glucose in the production of a retort cooked rice food. However, these documents disclose nothing about any improvement in physical properties. Furthermore, there has never been reported so far, not only for improving physical properties but also for any other purpose, to use glucose oxidase together with α-glucosidase and/or transglutaminase in a cooked rice food. A number of reports have been made on the use of glucose oxidase in breads and it is known that glucose oxidase has an effect of improving bread dough stability. However, cooked rice is a food that is completely different from bread and, therefore, the effect achieved by adding glucose oxidase to cooked rice would largely differ from those achieved by adding the same to bread.

### SUMMARY OF THE INVENTION

Accordingly, it is one object of the present invention to provide novel methods for producing a cooked rice food.

It is another object of the present invention to provide novel methods for producing a cooked rice food which has improved physical properties and palatability.

It is another object of the present invention to provide novel enzyme preparations which are useful for improving a cooked rice food.

It is another object of the present invention to provide novel enzyme preparations which are useful for preparing a cooked rice food.

It is another object of the present invention to provide novel methods for improving the qualities (palatability and physical properties) of a cooked rice food immediately after producing, preventing degradation in the qualities thereof with the passage of time during the production process and the distribution process after the production, and, furthermore, improving the processing suitability of processed cooked rice.

It is another object of the present invention to provide novel methods for producing a cooked rice food having such a texture including, for example, “glutinousness,” “puffiness,” “graininess,” and “non-clumping properties.”

These and other objects, which will become apparent during the following detailed description, have been achieved by the inventors' discovery that the aforesaid purposes can be achieved by using transglutaminase and/or α-glucosidase together with glucose oxidase.

Thus, the present invention provides:

(1) A method for producing a cooked rice food, which comprises using transglutaminase and/or α-glucosidase together with glucose oxidase.

(2) A method for producing a cooked rice food, which comprises treating rice with 0.0001 to 120 U of transglutaminase per g of uncooked rice as a raw material, and/or 0.03 to 300,000 U of α-glucosidase per g of uncooked rice as a raw material, together with 0.001 to 500 U of glucose oxidase per g of uncooked rice as a raw material.

(3) A method for producing a cooked rice food, which comprises treating rice with 0.05 to 12 U of transglutaminase per g of uncooked rice as a raw material, and/or 15 to 150,000 U of α-glucosidase per g of uncooked rice as a raw material, together with 0.03 to 210 U of glucose oxidase per g of uncooked rice as a raw material.

(4) The method according to any of (1) to (3), wherein glucose oxidase is added at a ratio of 0.003 to 10,000 U per U of transglutaminase and/or 0.000003 to 34 U per U of α-glucosidase.

(5) The method according to any of (1) to (4), wherein glucose oxidase is added at a ratio of 0.1 to 900 U per U of transglutaminase and/or 0.000006 to 3 U per U of α-glucosidase.

(6) An enzyme preparation for improving a cooked rice food, which comprises, as the active ingredients, transglutaminase and/or α-glucosidase together with glucose oxidase.

(7) The enzyme preparation according to (6), wherein the glucose oxidase content is at a ratio of 0.003 to 10,000 U per U of transglutaminase and/or 0.000003 to 34 U per U of α-glucosidase.

(8) The enzyme preparation according to (6) or (7), wherein the glucose oxidase content is at a ratio of 0.1 to 900 U per U of transglutaminase and/or 0.000006 to 3 U per U of α-glucosidase.

(9) A cooked rice food, which is prepared by a method according to any of (1) to (5).

The invention makes it possible to improve a cooked rice food. In particular, the qualities (physical properties such as puffiness, glutinousness, graininess and easy loosening properties) of a cooked rice food immediately after producing can be improved and the degradation in the qualities thereof with the passage of time can be prevented.

### BRIEF DESCRIPTION OF THE DRAWINGS

A more complete appreciation of the invention and many of the attendant advantages thereof will be readily obtained as the same become better understood by reference to the following detailed description when considered in connection with the accompanying drawings, wherein:

FIG. 1 shows the results of a sensory evaluation of frozen cooked rice (Example 1).

### DETAILED DESCRIPTION OF THE PREFERRED EMBODIMENTS

In the present method for producing a cooked rice food and the enzyme preparation for improving a cooked rice food according to the present invention, transglutaminase and/or α-glucosidase are used with glucose oxidase. That is, transglutaminase is used together with glucose oxidase, α-glucosidase is used together with glucose oxidase or transglutaminase and α-glucosidase are used together with glucose oxidase.

Glucose oxidase is an oxidizing enzyme catalyzing a reaction whereby gluconic acid and hydrogen peroxide are formed from glucose, oxygen and water that are employed as the substrates. Hydrogen peroxide that is formed by the reaction oxidizes an SH group in a protein and thus promotes the formation of an SS bond (a disulfide bond) to thereby form a cross-linked structure in the protein. There have been known glucose oxidases originating in various sources such as microorganisms, plants and so on. In the present invention, any enzyme having the aforesaid activity can be used regardless of its origin. Also, use can be made of a recombinant enzyme. As an example thereof, a microorganism-origin glucose oxidase available from Shinnihon Chemicals Co. under the trade name “SUMIZYME PGO” may be cited. There have been marketed glucose oxidase preparations also containing catalase. These mixtures of glucose oxidase with other enzymes are also usable in the invention so long as they have the glucose oxidase activity.

In the present invention, transglutaminase means an enzyme having an activity of catalyzing a transacylation reaction wherein a glutamine residue serves as a donor and a lysine residue serves as a receptor in a protein or peptide. There have been known transglutaminases originating in various sources such as mammals, fishes, microorganisms and so on. In the present invention, any enzyme having the aforesaid activity can be used regardless of its origin. Also, use can be made of a recombinant enzyme. As an example thereof, an actinomycete-origin transglutaminase available from Ajinomoto Co. under the trade name “ACTIVA TG” may be cited.

In the present invention, α-glucosidase means an enzyme hydrolyzing an α-1,4-glucoside bond at the non-reducing end to form α-glucose. Among α-glucosidases, transglucosidase having a transglycosylating activity from an α-1,4 bond to an α-1,6 bond is preferred. As an example of α-glucosidase, the enzyme available from Amano Enzyme Inc. under the trade name transglucosidase L “AMANO” may be cited.

Examples of the cooked rice food according to the invention include cooked rice (plain white rice), vinegar-seasoned rice (sushi meshi), adzuki bean rice, pilaf, fried rice, rice cooked with seasoned ingredients, cooked glutinous rice, rice gruel, risotto, rice ball, sushi, box lunch, and so on. Furthermore, the present invention includes in its scope products produced by freezing, aseptic-packing, retort pouch-packing, drying or canning these foods.

The rice to be used as the raw material of the cooked rice food according to the present invention may be of any variety. Either soft rice or hard rice may be used and either newly harvested rice or old rice may be used. Either low-quality rice or high-quality rice may be used. Furthermore, use may be made of processed rice having been treated with an acid or an enzyme such as low-protein rice (protein content-adjusted rice).

In the method for producing the cooked rice food according to the present invention, the treatment of rice with transglutaminase and/or α-glucosidase together with glucose oxidase may be conducted at any stage prior to cooking or after cooking. Namely, the enzymes may be added to soaking water in which rice is to be soaked to absorb water. Alternatively, the enzymes may be added at a point between soaking and cooking. It is also possible that, after cooking, the enzymes are sprinkled on cooked rice. In a production line for a steamed rice food, the enzymes may be sprinkled on rice before or after steaming. For treating rice, transglutaminase, α-glucosidase, and glucose oxidase may be used in an arbitrary order without restriction. The treatment with either one or two enzymes may be first conducted followed by the treatment with the residual one(s). In the case of using all of these three enzymes, however, it is preferred to treat rice with these three enzymes simultaneously. In addition, enzymes other than the aforesaid ones or other substances may be used together.

In the invention, glucose oxidase is added to treat rice at a ratio, in terms of enzyme activity, of 0.001 U or above, preferably 0.001 to 500 U, and more preferably 0.03 to 210 U, per g of uncooked rice as a raw material. The treatment of rice with glucose oxidase exerts an effect of imparting remarkable “glutinousness” and “elasticity” to the cooked rice food. In particular, such “glutinousness” that cannot be obtained by adding transglutaminase or α-glucosidase can be achieved thereby. Although “elasticity” at a certain degree can be obtained by using transglutaminase and α-glucosidase, the “elasticity” can be improved by increasing the ratio of transglutaminase. In this case, however, the effect of α-glucosidase of imparting “puffiness” is somewhat reduced. By adding glucose oxidase, in contrast thereto, strong “elasticity” can be imparted without undesirably inhibiting the function of α-glucosidase. When 500 U or more glucose oxidase is added, the “elasticity” is excessively elevated, and the resultant cooked rice food shows an unnatural gummy texture and, therefore, becomes less preferable.

The enzyme activity of glucose oxidase is determined by treating glucose, as a substrate, with glucose oxidase in the presence of oxygen to form hydrogen peroxide, treating the thus formed hydrogen peroxide with peroxidase in the presence of aminoantipyrine and phenol, and measuring the color tone of the quinone imine coloring matter thus formed at a wavelength of 500 nm. One unit (U) of the enzyme is defined as the amount of the enzyme required for oxidizing 1 μmol of glucose in 1 minute. The activity of glucose oxidase may be measured as described in Voluntary Specifications of Existing Food Additives, Fourth edition, Japan Food Additive Association, p. 91, October 113, 2008, which is incorporated herein by reference in its entirety.

In the invention, when glucose oxidase is added at a ratio, in terms of enzyme activity, of 0.001 U or above, preferably 0.001 to 500 U and more preferably 0.03 to 210 U, per g of uncooked rice, transglutaminase is added to treat rice at a ratio, in terms of enzyme activity, of 0.0001 U or above, preferably 0.0001 to 120 U and more preferably 0.05 to 12 U, per g of uncooked rice. It is also desirable that transglutaminase is added in such an amount as to control the amount of added glucose oxidase to 0.003 to 10,000 U, preferably 0.1 to 900 U, per U of transglutaminase.

The enzyme activity of transglutaminase is determined by reacting benzyloxycarbonyl-L-glutaminyl glycine and hydroxylamine that are employed as substrates, forming an iron complex of the thus formed hydroxamic acid in the presence of trichloroacetic acid, measuring the absorbance at 525 nm, and then determining the amount of hydroxamic acid from a calibration curve to thereby calculate the activity. One unit (U) of the enzyme is defined as the amount of the enzyme required for forming 1 μmol of hydroxamic acid in 1 minute at 37° C. and pH 6.0. The activity of transglutaminase may be measured as described in U.S. Pat. No. 5,156,956 or Food Reviews International, vol. 17(2), pp. 221-246 (2001), which are incorporated herein by reference in their entireties.

In the invention, α-glucosidase is added to treat rice at a ratio, in terms of enzyme activity, of 0.03 U or above, preferably 0.03 to 300,000 U and more preferably 15 to 150,000 U, per g of uncooked rice as a raw material. It is also desirable that α-glucosidase is added in such an amount as controlling the amount of added glucose oxidase to 0.000003 to 34 U, preferably 0.000006 to 3 U, per U of α-glucosidase.

One unit (U) of α-glucosidase is defined as the amount of the enzyme required for forming 1 μg of glucose in 2.5 ml of a liquid reaction mixture, in the case of adding 1 ml of a 0.02 M acetate buffer (pH 5.0) to 1 ml of 1 mM α-methyl-D-glucoside, adding 0.5 ml of an enzyme solution thereto and then reacting at 40° C. for 60 minutes. The activity of α-glucosidase may be measured as described in US 2007/0110847, which is incorporated herein by reference in its entirety.

As stated above, in the case of producing a cooked rice food by treating rice with transglutaminase and/or α-glucosidase together with glucose oxidase, it is appropriate to add the enzymes at the ratios, in terms of enzyme activity (U), 0.003 to 10,000 U of glucose oxidase, preferably 0.1 to 900 U of glucose oxidase per U of transglutaminase, and/or 0.000003 to 34 U, preferably 0.000006 to 3 U of glucose oxidase per U of α-glucosidase. When these enzymes are added respectively in the amounts as defined above, it is possible to produce a cooked rice food which has excellent qualities (i.e., properties such as puffiness, glutinousness, graininess and easy loosening properties) immediately after the production and suffers from less degradation in the qualities with the passage of time.

The reaction time of each enzyme is not particularly restricted so long as the enzymatic treatment of the substrate can be completed therein. Although an extremely short or long reaction time may be employed, it is preferred from a practical viewpoint to conduct the treatment for 5 minutes to 24 hours. Similarly, the reaction temperature may be arbitrarily selected so long as the enzyme can maintain the activity thereof. From a practical viewpoint, it is preferred to carry out the treatment at 0 to 80° C. That is, a sufficient reaction time can be obtained by a method commonly employed for cooking rice.

An enzyme preparation for improving a cooked rice food can be obtained by blending transglutaminase and/or α-glucosidase and glucose oxidase with food additives, for example, a bulking agent such as dextrin, starch, a processed starch or reducing maltose, a seasoning such as a meat extract, a protein such as a vegetable protein, gluten, egg albumen, gelatin or casein, a hydrolyzed protein, a partially hydrolyzed protein, an emulsifier, a chelating agent such as a citric acid salt or a polyphosphoric acid salt, a reducing agent such as glutathione or cysteine, alginic acid, alkaline water, fat or oil, a dye, a souring agent, a flavor and so on. The enzyme preparation according to the present invention may be in any form such as a liquid, a paste, granules or a powder. The contents of the individual enzymes in the enzyme preparation are each more than 0% and less than 100%. It is desirable that the glucose oxidase content is 0.003 to 10,000 U, preferably 0.1 to 900 U per U of transglutaminase, and/or 0.000003 to 34 U, preferably 0.000006 to 3 U per U of α-glucosidase.

Other features of the invention will become apparent in the course of the following descriptions of exemplary embodiments which are given for illustration of the invention and are not intended to be limiting thereof.

### EXAMPLES

### Example 1

300 g of uncooked rice “Hokkaido Kirara 397” was washed with tap water and soaked in tap water for one hour. After draining, the rice was supplied into an IH jar rice cooker Model NJ-HS06-S (manufactured by Mitsubishi Electric Co.). Next, tap water was added thereto so as to give a water amount 1.5 times as much as the uncooked rice amount. Namely, tap water was added in such an amount that the sum of the water absorbed by the uncooked rice during soaking and the amount of water added after soaking attained 450 g, i.e., 1.5 times as much as the amount of the uncooked rice (300 g). Then, “Transglucosidase L” (manufactured by Amano Enzyme Inc.; hereinafter referred to as TGL) that is α-glucosidase having a transglycosylating activity of converting an α-1,4 bond to an α-1,6 bond, a transglutaminase preparation “ACTIVA TG” (manufactured by Ajinomoto Co., hereinafter referred to as TG) and a glucose oxidase preparation “SUMIZYME PGO” (hereinafter referred to as GO) were added thereto and dissolved therein. The obtained mixture was cooked with the aforesaid rice cooker. Table 1 shows the amounts of the individual enzymes added.

TABLE 1
Amount of enzymes added in cooking rice.

| | Amount of preparation | | | | |
|---|---|---|---|---|---|
| | added | | | Addition ratio of | |
| | TGL | TG | GO | preparation | |
| | (U/g of | (U/g of | (U/g of | GO per | GO per |
| | uncooked | uncooked | uncooked | TGL 1U | TG 1U |
| | rice) | rice) | rice) | (U) | (U) |
| Cont (no | — | — | — | — | — |
| addition) | | | | | |
| 1) TGL | 70.69 | — | — | — | — |
| 2) TG | — | 0.26 | — | — | — |
| 3) GO | — | — | 0.95 | — | — |
| 4) TGL + TG | 70.69 | 0.26 | — | — | — |
| 5) TGL + GO | 70.69 | — | 0.95 | 0.013 | — |
| 6) TG + GO | — | 0.26 | 0.95 | — | 3.65 |
| 7) TGL + TG + | 70.69 | 0.26 | 0.95 | 0.013 | 3.65 |
| GO | | | | | |
| 8) TGL + TG + | 70.69 | 0.17 | 1.68 | 0.024 | 9.68 |
| GO | | | | | |

Each cooked rice food was quickly frozen at −40° C. in a quick freezer “Blast Chiller/Shock Freezer Model QXF-012SF5” (manufactured by Fukushima Industries, Co.) to give a frozen cooked rice. After storing for one week at −20° C., the frozen cooked rice was microwaved and subjected to sensory evaluation. Table 2 shows the results. The sensory evaluation was conducted for eight items including hardness, glutinousness, water-retention, puffiness, stickiness, non-clumping properties, elasticity and graininess. Moreover, preference in using Japanese rice dishes and preference in using Western/Chinese rice dishes were given as total evaluation. In the sensory evaluation items, “hardness” means the strength of a stress felt in grinding with the teeth; “glutinousness” means a glutinous texture felt around the teeth in chewing; “water-retention” means showing no water-release from inside in chewing; “puffiness” means a complex feature consisting of “softness” (the opposite of “hardness”), “glutinousness” and “water-retention”; “stickiness” means the adhesiveness of grain surface; “non-clumping properties” means that grains can be loosened in the mouth without sticking together; “elasticity” means the strength of a repulsion force, i.e., restoration, in chewing; and “graininess” means a complex feature consisting of “non-clumping properties” and “elasticity.” Japanese rice dishes include plain white rice, rice mixed with seasoned ingredients, etc., while Western/Chinese rice dishes include pilaf, fried rice, etc. Although the evaluation items should be well-balanced in both of Japanese rice dishes and Western/Chinese rice dishes, a texture with strong “glutinousness” and “puffiness” is preferred in Japanese rice dishes, while a texture with strong “elasticity” and “graininess” is preferred in Western/Chinese rice dishes. The evaluation was made by five panelists and scored in the −3 to +3 scale, referring to a sample in a control lot, where no enzyme was added, as to 0. The total evaluation was made by calculating theoretical scores in test lots where two or three enzymes were used together, based on the results of test lots where TGL alone, TG alone, and GO alone were respectively added. For example, the theoretical score of sample 7) to be used in Japanese rice dishes was calculated as follows. Since the score of the TGL (70.69 U/g)-alone lot was 1 and 70.69 U/g of TGL was used in sample 7), the theoretical score of this lot was expressed in: 1×70.69/70.69=1. Since the score of the TG (0.26 U/g)-alone lot was 0 and 0.17 U/g of TG was used in sample 7), the theoretical score of this lot was expressed in: 0×0.17/0.26=0. Since the score of the GO (0.95 U/g)-alone lot was 0.5 and 1.68 U/g of GO was used in sample 7), the theoretical score of this lot was expressed in: 0.5×1.68/0.95=0.884. Thus, the theoretical score of sample 7) to be used in Japanese rice dishes was calculated as: 1+0+0.884=1.884. Therefore, “1.884” is a theoretical value of total evaluation using Japanese rice dishes. The theoretical value thus calculated (“1.884” in this case) is compared with the practical score (“3” in this case). When these scores are the same, it can be understood that the theoretical effect, i.e., an additive effect is established. When the practical score is larger than the theoretical score, synergistic effects are established. Since the practical score “3” exceeds the theoretical score “1.884” in the above case, it can be concluded that synergistic effects were established in this case.

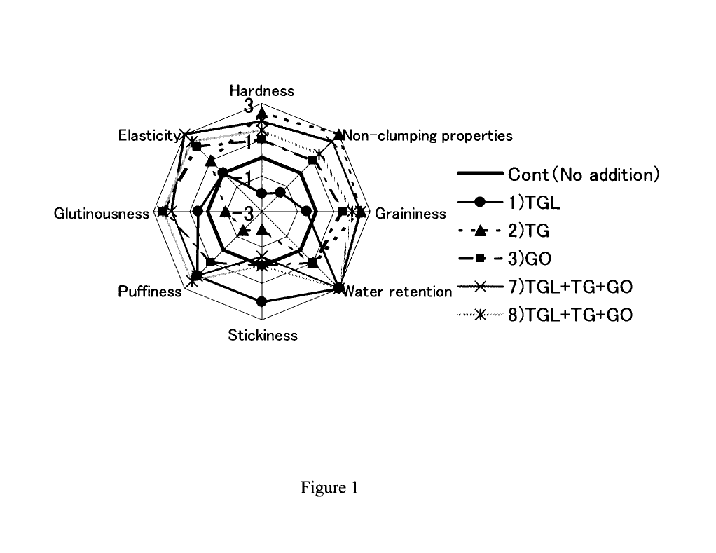

As Table 2 and FIG. 1 show, the addition of TGL alone mainly imparted “puffiness,” “stickiness,” and “water-retention”; the addition of TG alone mainly imparted “hardness,” “non-clumping properties,” and “graininess”; the addition of GO alone mainly imparted “glutinousness” and “elasticity.” Thus, it can be understood that all of the eight evaluation items can be controlled with the use of these three enzymes. In the case of using TGL and TG together, effects of imparting “hardness” and remarkable “water-retention” were observed but the effect of imparting “puffiness” was weak and no “glutinousness” was imparted. In the case of using TGL together with GO, only weak effects of imparting “hardness,” “non-clumping properties,” and “graininess” were observed but remarkable “glutinousness,” “water-retention,” and “puffiness” were imparted, thereby giving a texture suitable for Japanese rice dishes. In the total evaluation, the practical scores exceeded the theoretical scores both in Japanese rice dishes and Western/Chinese rice dishes, which indicates that the combined use of TGL with GO exerted synergistic effects. In the case of using TG and GO together, no effect of imparting “puffiness” was observed but remarkable “hardness,” “non-clumping properties,” “elasticity,” and “graininess” were imparted, thereby giving a texture suitable for Western/Chinese rice dishes such as pilaf and fried rice. In the total evaluation, the practical score exceeded the theoretical score in Western/Chinese rice dishes, which indicates that the combined use of TG with GO exerted synergistic effects. In 7) and 8) with the use of the three enzymes together, well-balanced effects were observed in each of the eight items. In the total evaluation, the practical scores of 7) and 8) both exceeded the theoretical scores both in Japanese rice dishes and Western/Chinese rice dishes, which indicates that the combined use of TGL and TG with GO exerted synergistic effects. With respect to “stickiness,” the samples were cooked with water in an amount of giving sufficient “stickiness” and, therefore, no increase in “stickiness” by the addition of the enzymes is required any more. As discussed above, it was clarified that these three enzymes, i.e. TGL, TG and GO exert synergistic effects and give a preferable texture, when used in combination.

TABLE 2
Sensory evaluation results of frozen cooked rice.

| | Sensory evaluation score (point) | | | | | | | | | | | | Theoretical score | |
|---|---|---|---|---|---|---|---|---|---|---|---|---|---|---|
| | | | Water | | | | | | Total evaluation | | | | Total evaluation | |
| | | | retention | | | Non- | | | (preference) | | | | (preference) | |
| | Hard- | Gluti- | (no water | | Stickiness | clumping | Elas- | | | | Western/ | | | Western/ |
| | ness | nousness | liberation) | Puffiness | (surface) | properties | ticity | Graininess | Japanese | | Chinese | | Japanese | Chinese |
| Cont | 0 | 0 | 0 | 0 | 0 | 0 | 0 | 0 | 0 | X | 0 | X | — | — |
| (no addition) | | | | | | | | | | | | | | |
| 1) TGL | −2 | 0.5 | 3 | 2 | 2 | −1.5 | 0 | −0.5 | 1 | Δ | 0 | X | — | — |
| 2) TG | 2.5 | −1 | 1 | −1.5 | −2 | 3 | 1 | 2.5 | 0 | X | 0.5 | X | — | — |
| 3) GO | 1 | 2.5 | 1 | 1 | 0 | 1 | 2 | 1.5 | 0.5 | X | 0.5 | X | — | — |
| 4) TGL + TG | 2 | −0.5 | 3 | 0.5 | −0.5 | 1.5 | 1 | 1.5 | 1 | Δ | 1 | Δ | 1 | 0.5 |
| 5) TGL + GO | 0.5 | 3 | 3 | 3 | 2 | 0.5 | 1.5 | 1 | 2 | ◯ | 1 | Δ | 1.5 | 0.5 |
| 6) TG + GO | 3 | 1.5 | 1.5 | 0 | −1.5 | 3 | 3 | 3 | 0.5 | X | 1.5 | ◯ | 0.5 | 1 |
| 7) TGL + | 2 | 2 | 3 | 2 | −0.5 | 2.5 | 3 | 2.5 | 2.5 | ◯ | 3 | ◯◯ | 1.5 | 1 |
| TG + GO | | | | | | | | | | | | | | |
| 8) TGL + | 1.5 | 2.5 | 3 | 2.5 | 0 | 1.5 | 2.5 | 2 | 3 | ◯ | 2.5 | ◯◯ | 1.9 | 1.2 |
| TG + GO | | | | | | | | | | | | | | |

### Example 2

By using the enzymes, frozen cooked rice were obtained as in Example 1. Table 3 shows the ratios and amounts of the enzymes used. After storing for one week at −20° C., each frozen cooked rice was microwaved and subjected to sensory evaluation. The sensory evaluation was conducted for eight items including hardness, glutinousness, water-retention, puffiness, stickiness, non-clumping properties, elasticity, and graininess. Then, the preference of texture balance as plain white rice was expressed in the symbols oo, o, Δ, and x. oo means “highly preferred,” o means “preferred,” Δ means “somewhat preferred,” and x means not preferred.” The evaluation was made by five panelists. In Table 3, “3.4E+2,” “5.6E−6” and so on indicate indexes and respectively mean “3.4×10²,” “5.6×10⁻⁶” and the like.

TABLE 3
Sensory evaluation results of frozen cooked rice.

| | Test lot | | | | | | | | | | |
|---|---|---|---|---|---|---|---|---|---|---|---|
| | 1 | 2 | 3 | 4 | 5 | 6 | 7 | 8 | 9 | 10 | 11 |
| TGL (U/g of | 0 | 0.003 | 0.03 | 15 | 38 | 45 | 50 | 75 | 88 | 95 | 100 |
| uncooked rice) | | | | | | | | | | | |
| TG (U/g of | 0 | 0.26 | 0.26 | 0.26 | 0.26 | 0.26 | 0.26 | 0.26 | 0.26 | 0.26 | 0.26 |
| uncooked rice) | | | | | | | | | | | |
| GO (U/g of | 0 | 0.84 | 0.84 | 0.84 | 0.84 | 0.84 | 0.84 | 0.84 | 0.84 | 0.84 | 0.84 |
| uncooked rice)¹ | | | | | | | | | | | |
| GO/TGL | 0 | 3.4E+02 | 3.4E+01 | 5.6E−02 | 2.2E−02 | 1.9E−02 | 1.7E−02 | 1.1E−02 | 9.6E−03 | 8.8E−03 | 8.4E−03 |
| (ratio by U) | | | | | | | | | | | |
| GO/TG | 0 | 3.2E+00 | 3.2E+00 | 3.2E+00 | 3.2E+00 | 3.2E+00 | 3.2E+00 | 3.2E+00 | 3.2E+00 | 3.2E+00 | 3.2E+00 |
| (ratio by U) | | | | | | | | | | | |
| Sensory | X | X | Δ | ◯ | ◯◯ | ◯◯ | ◯◯ | ◯◯ | ◯◯ | ◯◯ | ◯◯ |
| evaluation | | | | | | | | | | | |
| result | | | | | | | | | | | |
| | Test lot | | | | | | | | | | |
| | 12 | 13 | 14 | 15 | 16 | 17 | 18 | 19 | 20 | 21 | 22 |
| TGL (U/g of | 5000 | 150000 | 300000 | 375000 | 70.8 | 70.8 | 70.8 | 70.8 | 70.8 | 70.8 | 70.8 |
| uncooked rice) | | | | | | | | | | | |
| TG (U/g of | 0.26 | 0.26 | 0.26 | 0.26 | 0.00005 | 0.0001 | 0.05 | 0.14 | 0.16 | 0.18 | 0.23 |
| uncooked rice) | | | | | | | | | | | |
| GO (U/g of | 0.84 | 0.84 | 0.84 | 0.84 | 0.84 | 0.84 | 0.84 | 0.84 | 0.84 | 0.84 | 0.84 |
| uncooked rice)¹ | | | | | | | | | | | |
| GO/TGL | 1.7E−04 | 5.6E−06 | 2.8E−06 | 2.2E−06 | 1.2E−02 | 1.2E−02 | 1.2E−02 | 1.2E−02 | 1.2E−02 | 1.2E−02 | 1.2E−02 |
| (ratio by U) | | | | | | | | | | | |
| GO/TG | 3.2E+00 | 3.2E+00 | 3.2E+00 | 3.2E+00 | 1.8E+04 | 9.1E+03 | 1.8E+01 | 6.1E+00 | 5.2E+00 | 4.6E+00 | 3.7E+00 |
| (ratio by U) | | | | | | | | | | | |
| Sensory | ◯ | ◯ | Δ | X | X | Δ | ◯ | ◯◯ | ◯◯ | ◯◯ | ◯◯ |
| evaluation | | | | | | | | | | | |
| result | | | | | | | | | | | |
| | Test lot | | | | | | | | | | |
| | 23 | 24 | 25 | 26 | 27 | 28 | 29 | 30 | 31 | 32 | 33 |
| TGL (U/g of | 70.8 | 70.8 | 70.8 | 70.8 | 70.8 | 70.8 | 70.8 | 70.8 | 70.8 | 70.8 | 70.8 |
| uncooked rice) | | | | | | | | | | | |
| TG (U/g of | 0.32 | 0.35 | 0.37 | 0.41 | 11.5 | 115 | 230 | 0.26 | 0.26 | 0.26 | 0.26 |
| uncooked rice) | | | | | | | | | | | |
| GO (U/g of | 0.84 | 0.84 | 0.84 | 0.84 | 0.84 | 0.84 | 0.84 | 0.0001 | 0.001 | 0.03 | 0.42 |
| uncooked rice)¹ | | | | | | | | | | | |
| GO/TGL | 1.2E−02 | 1.2E−02 | 1.2E−02 | 1.2E−02 | 1.2E−02 | 1.2E−02 | 1.2E−02 | 1.2E−06 | 1.2E−05 | 3.6E−04 | 5.9E−03 |
| (ratio by U) | | | | | | | | | | | |
| GO/TG | 2.6E+00 | 2.4E+00 | 2.3E+00 | 2.0E+00 | 7.3E−02 | 7.3E−03 | 3.7E−03 | 3.2E−04 | 3.2E−03 | 9.7E−02 | 1.6E+00 |
| (ratio by U) | | | | | | | | | | | |
| Sensory | ◯◯ | ◯◯ | ◯◯ | ◯◯ | ◯ | Δ | X | X | Δ | ◯ | ◯◯ |
| evaluation | | | | | | | | | | | |
| result | | | | | | | | | | | |
| | Test lot | | | | | | | | | | |
| | 34 | 35 | 36 | 37 | 38 | 39 | 40 | 41 | 42 | 43 | 44 |
| TGL (U/g of | 70.8 | 70.8 | 70.8 | 70.8 | 70.8 | 70.8 | 70.8 | 70.8 | 70.8 | 70.8 | 70.7 |
| uncooked rice) | | | | | | | | | | | |
| TG (U/g of | 0.26 | 0.26 | 0.26 | 0.26 | 0.26 | 0.26 | 0.26 | 0.26 | 0.26 | 0.26 | 0.17 |
| uncooked rice) | | | | | | | | | | | |
| GO (U/g of | 0.50 | 0.59 | 0.67 | 1.18 | 1.26 | 1.34 | 1.68 | 210 | 504 | 3360 | 1.68 |
| uncooked rice)¹ | | | | | | | | | | | |
| GO/TGL | 7.1E−03 | 8.3E−03 | 9.5E−03 | 1.7E−02 | 1.8E−02 | 1.9E−02 | 2.4E−02 | 3.0E+00 | 7.1E+00 | 4.7E+01 | 2.4E−02 |
| (ratio by U) | | | | | | | | | | | |
| GO/TG | 1.9E+00 | 2.3E+00 | 2.6E+00 | 4.5E+00 | 4.8E+00 | 5.2E+00 | 6.5E+00 | 8.1E+02 | 1.9E+03 | 1.3E+04 | 9.7E+00 |
| (ratio by U) | | | | | | | | | | | |
| Sensory | ◯◯ | ◯◯ | ◯◯ | ◯◯ | ◯◯ | ◯◯ | ◯◯ | ◯ | Δ | X | ◯◯ |
| evaluation | | | | | | | | | | | |
| result | | | | | | | | | | | |

As Table 3 shows, when the three enzymes were used together, preferred effects were observed by adding TGL, TG and GO each in an amount falling within a specific range. Also, preferred effects were observed when the GO/TGL ratio (by U) and GO/TG ratio (by U) were each within a specific range. TGL exerted a somewhat preferred effect at an addition ratio of 0.03 to 300,000 U and a preferred effect at 15 to 150,000 U, each per g of uncooked rice. TG exerted a somewhat preferred effect at an addition ratio of 0.0001 to 115 U and a preferred effect at 0.05 to 11.5 U, each per g of uncooked rice. GO exerted a somewhat preferred effect at an addition ratio of 0.001 to 504 U and a preferred effect at 0.03 to 210 U, each per g of uncooked rice. With respect to the ratio by U of GO to TGL, a somewhat preferred effect was observed at 0.000003 to 34 U and a preferred effect was observed at 0.000006 to 3 U. With respect to the ratio by U of GO to TG, a somewhat preferred effect was observed at 0.003 to 9130 U and a preferred effect was observed at 0.1 to 807 U. These results can be summarized as follows. In the case of adding TGL, TG and GO to a cooked rice food, it is preferable to add TGL in an amount of 0.03 to 300,000 U, more preferably 15 to 150,000 U, each per g of uncooked rice. It is preferable to add TG in an amount of 0.0001 to 120 U, more preferably 0.05 to 12 U, each per g of uncooked rice. It is preferable to add GO in an amount of 0.001 to 500 U, more preferably 0.03 to 210 U, each per g of uncooked rice. The ratio by U of GO to TGL is preferably 0.000003 to 34 U, more preferably 0.000006 to 3 U. The ratio by U of GO to TG is preferably 0.003 to 10,000 U, more preferably 0.1 to 900 U. By using the three enzymes together in the aforesaid ranges, preferable “hardness,” “glutinousness,” “water-retention,” “puffiness,” “stickiness,” “non-clumping properties,” and “graininess” can be achieved in a well-balanced manner without compromising any of these items. In particular, the simultaneous imparting of both a “hard” and “grainy” feel and a “glutinous” and “puffy” feel, which has been considered as difficult so far, contributes to the achievement of a preferable texture.

### Example 3

By using the enzymes, frozen cooked rice foods were obtained as in Example 1. The enzyme composition was fixed based on the composition (7 in Table 1) employed in Example 1 whereby preferable texture was obtained. The enzymes were added in the amounts listed in Table 4. After storing for one week at −20° C., each frozen cooked rice was microwaved and subjected to sensory evaluation. The sensory evaluation was made for eight items including hardness, glutinousness, water-retention, puffiness, stickiness, non-clumping properties, elasticity, and graininess. The preference as plain white rice was expressed in the symbols oo, o, Δ, and x. oo means “highly preferred,” o means “preferred,” Δ means “somewhat preferred,” and x means “not preferred.” The evaluation was made by four panelists.

TABLE 4
Amounts of enzymes added in cooking rice.

| | Test lot | | | | | | | | | | |
|---|---|---|---|---|---|---|---|---|---|---|---|
| | Control | 1 | 2 | 3 | 4 | 5 | 6 | 7 | 8 | 9 | 10 |
| TGL (U/g of | 0 | 0.071 | 0.71 | 7.07 | 70.69 | 1413.8 | 3534.5 | 7069 | 14138 | 35345 | 70690 |
| uncooked rice) | | | | | | | | | | | |
| TG (U/g of | 0 | 0.00026 | 0.0026 | 0.026 | 0.26 | 5.20 | 13.0 | 26.0 | 52.0 | 130 | 260 |
| uncooked rice) | | | | | | | | | | | |
| GO (U/g of | 0 | 0.0010 | 0.010 | 0.10 | 0.95 | 19.0 | 47.50 | 95.0 | 190 | 475.0 | 950 |
| uncooked rice)¹ | | | | | | | | | | | |
| GO/TGL | 0 | 0.013 | 0.013 | 0.013 | 0.013 | 0.013 | 0.013 | 0.013 | 0.013 | 0.013 | 0.013 |
| (ratio by U) | | | | | | | | | | | |
| GO/TG | 0 | 3.65 | 3.65 | 3.65 | 3.65 | 3.65 | 3.65 | 3.65 | 3.65 | 3.65 | 3.65 |
| (ratio by U) | | | | | | | | | | | |
| Sensory evaluation | X | X | Δ | ◯ | ◯◯ | ◯ | ◯ | ◯ | Δ | X | X |
| result | | | | | | | | | | | |

As Table 4 shows, TGL exerted a somewhat preferred effect at an addition ratio of 0.71 to 14,138 U and a preferred effect at 7.07 to 7.069 U, each per g of uncooked rice. TG exerted a somewhat preferred effect at an addition ratio of 0.0026 to 52 U and a preferred effect at 0.026 to 26 U, each per g of uncooked rice. GO exerted a somewhat preferred effect at an addition ratio of 0.01 to 190 U and a preferred effect at 0.1 to 95 U, each per g of uncooked rice. When the enzymes were added in excessively large amounts, the strengthened hardness caused deterioration in texture balance. When the enzymes were added in excessively small amounts, the effects of the enzymes were limited and, therefore, no preferred effect was obtained.

### Example 4

300 g of uncooked rice “Hokkaido Kirara 397” was washed with tap water and soaked in tap water for one hour. After draining, the rice was supplied into an IH jar rice cooker Model NJ-HS06-S (manufactured by Mitsubishi Electric Co.). Next, tap water was added so as to give a water amount 1.5 times as much as the uncooked rice amount. Then, TGL, TG and GO were added thereto and dissolved, followed by cooking with the rice cooker. Table 5 shows the amounts of the enzymes. After adding a vinegar mixture in an amount of 10 wt % relative to the cooked rice thus obtained, the sushi rice thus obtained was molded in 25 g portions and quickly frozen at −40° C. in a quick freezer “Blast Chiller/Shock Freezer Model QXF-012SF5” (manufactured by Fukushima Industries, Co.) to give a frozen sushi rice. The vinegar mixture comprised 63% of cereal vinegar, 31% of refined sucrose and 6% of sodium chloride. After storing for one week at −20° C., the frozen sushi rice was spontaneously thawed by allowing to stand for 3 hours at room temperature and then subjected to sensory evaluation. Table 5 shows the results. The sensory evaluation was made for eight items including hardness, glutinousness, water-retention, puffiness, stickiness, non-clumping properties, elasticity, and graininess. Moreover, preference in texture balance as sushi was expressed in the symbols oo, o, Δ, and x. oo means “highly preferred,” o means “preferred,” Δ means “somewhat preferred,” and x means “not preferred.” The evaluation was made by four panelists.

TABLE 5
Amounts of enzymes added in cooking rice and sensory evaluation results of frozen sushi rice.

| | Test lot | | | | | | | |
|---|---|---|---|---|---|---|---|---|
| | 1 | 2 | 3 | 4 | 5 | 6 | 7 | 8 |
| TGL (U/g of uncooked rice) | — | 312.50 | — | — | 70.69 | 70.69 | 141.38 | 141.38 |
| TG (U/g of uncooked rice) | — | — | 0.58 | — | 0.26 | 0.13 | 0.13 | — |
| GO (U/g of uncooked rice)¹ | — | — | — | 1.05 | 0.95 | 0.95 | 0.95 | 0.95 |
| GO/TGL | — | — | — | — | 0.013 | 0.013 | 0.007 | 0.007 |
| (ratio by U) | | | | | | | | |
| GO/TG | — | — | — | — | 3.654 | 7.308 | 7.308 | — |
| (ratio by U) | | | | | | | | |
| Sensory evaluation result | X | X | X | X | ◯◯ | ◯◯ | ◯◯ | ◯ |

As Table 5 shows, highly preferred effects were observed in the test lots where TGL, TG, and GO were used together, and a preferred effect was observed in the test lot where TGL and GO were used together, which suggests that the combined use of these enzymes enables the production of frozen sushimeshi having freeze resistance and a preferable texture.

### Example 5

25 g of uncooked rice “Koshihikari” was washed with tap water and drained for 60 minutes. Then, the rice was put into a retort pouch and tap water was added thereto to give a total weight including the uncooked rice of 252 g. After adding the enzymes, the pouch was sealed. Table 6 shows the amounts of the enzymes. After gently stirring and allowing to stand at room temperature for 60 minutes, the pouch was subjected to retort sterilization at 102° C. for 10 minutes and then at 121° C. until the F value attained 8 to give a retorted gruel. The retorted gruel was stored for two weeks at 20° C., then heated in boiling water for five minutes and subjected to sensory evaluation. Table 6 shows the results. The sensory evaluation was made by paying attention to hardness, puffiness, graininess, glutinousness (viscosity), etc. and the preference of texture balance as gruel was expressed in the symbols oo, o, Δ, and x. oo means “highly preferred,” o means “preferred,” Δ means “somewhat preferred,” and x means “not preferred.”. The evaluation was made by five panelists.

TABLE 6
Amounts of enzymes added in producing retort gruel and sensory evaluation results.

| | | Test lot | | | | |
|---|---|---|---|---|---|---|
| | Test lot | 1 | 2 | 3 | 4 | 5 |
| TGL (U/g of uncooked rice) | TGL added*¹ | — | 312.50 | — | — | 70.69 |
| TG (U/g of uncooked rice) | TG added*¹ | — | — | 0.58 | — | 0.26 |
| GO (U/g of uncooked rice)¹ | GO added*¹ | — | — | — | 1.05 | 0.95 |
| GO/TGL | GO/TGL ratio by U | — | — | — | — | 0.013 |
| (ratio by U) | | | | | | |
| GO/TG | GO/TG ratio by U | — | — | — | — | 3.654 |
| (ratio by U) | | | | | | |
| Sensory evaluation result | Sensory evaluation result | X | X | X | X | ◯◯ |

As Table 6 shows, a highly preferred effect was observed in the test lot where TGL, TG and GO were used together, which suggests that the combined use of these enzymes enables the production of retort gruel having retort resistance and a preferable texture. Also, an effect of preventing the rice grains from breakage was observed.

### Example 6

A non-glutinous rice variety “Hokkaido Kirara 397” and a glutinous rice variety “Mochigome” (available from KITOKU-SHINRYO Co., Ltd.) were blended at each ratio listed in Table 7, washed with tap water and soaked in tap water for one hour. Then, the rice having been soaked was drained and supplied into an IH jar rice cooker Model NJ-HS06-S (manufactured by Mitsubishi Electric Co.). Next, tap water was added so as to give a water amount 1.3 times or 1.45 times as much as the uncooked rice amount. Also, a retort-packed adzuki bean product for adzuki bean rice “Osekihan no moto” (manufactured by Imuraya Co., Ltd.) was added thereto. Further, TGL, TG, and GO were added thereto and dissolved, followed by cooking with the aforesaid rice cooker. Table 7 shows the amounts of the enzymes. Each adzuki bean rice thus cooked was quickly frozen at −40° C. in a quick freezer “Blast Chiller/Shock Freezer Model QXF-012SF5” (manufactured by Fukushima Industries, Co.) to give a frozen adzuki bean rice. After storing for one week at −20° C., the adzuki bean rice was microwaved and subjected to sensory evaluation. Table 7 shows the results. The sensory evaluation was made for eight items including hardness, glutinousness, water-retention, puffiness, stickiness, non-clumping properties, elasticity, and graininess. Moreover, preference in texture balance as adzuki bean rice was expressed in the symbols oo, o, Δ, and x. oo means “highly preferred,” o means “preferred,” Δ means “somewhat preferred,” and x means “not preferred.” The evaluation was made by four panelists. In a preferred texture as glutinous rice, it is seemingly highly important to have both glutinousness and stickiness.

TABLE 7
Amounts of enzymes added in producing frozen adzuki bean rice and sensory evaluation results thereof.

| | Test lot | | | | | | | | |
|---|---|---|---|---|---|---|---|---|---|
| | 1 | 2 | 3 | 4 | 5 | 6 | 7 | 8 | 9 |
| Non-Glutinous rice (%) | 60 | 80 | 80 | 80 | 80 | 80 | 80 | 80 | 80 |
| Glutinous rice (%) | 40 | 20 | 20 | 20 | 20 | 20 | 20 | 20 | 20 |
| Water Addition ratio | 1.3 | 1.45 | 1.45 | 1.45 | 1.45 | 1.45 | 1.45 | 1.45 | 1.45 |
| | times | times | times | times | times | times | times | times | times |
| TGL (U/g of uncooked rice) | — | — | — | 62.50 | 187.50 | 312.50 | 312.50 | 70.69 | 70.69 |
| TG (U/g of uncooked rice) | — | — | — | — | — | — | — | 0.17 | 0.26 |
| GO (U/g of uncooked rice)¹ | — | — | 1.68 | 1.68 | 1.68 | 1.68 | 2.52 | 1.68 | 0.95 |
| GO/TGL | — | — | — | — | 0.009 | 0.005 | 0.008 | 0.024 | 0.013 |
| (ratio by U) | | | | | | | | | |
| GO/TG | — | — | — | — | — | — | — | 9.882 | 3.654 |
| (ratio by U) | | | | | | | | | |
| Sensory evaluation result | ◯◯ | X | X | ◯◯ | ◯◯ | ◯◯ | ◯◯ | ◯ | ◯ |

As Table 7 shows, highly preferred effects were observed in the test lots where TGL and GO were used together, and highly preferred effects were observed in the test lot where TGL, TG, and GO were used together, which suggests that the combined use of these enzymes enables the achievement of a preferred texture of adzuki bean rice even in the case of using glutinous rice at a lower ratio. Although the adzuki bean rice sample of test lot 1, wherein the glutinous rice was used at a high ratio, showed a preferred texture immediately after microwaving, its texture became gummy and hard after returning to room temperature. In contrast thereto, the adzuki bean rice samples of test lots 4 to 9, which had been treated with the enzymes, maintained preferred texture (sticky and glutinous) of adzuki bean rice after returning to room temperature. These results suggest that the use of the enzymes is also effective in preventing these products from deterioration with the passage of time.

### Example 7

Two non-Japanese rice varieties (produced in USA), i.e., “SUSHI RICE” (medium-grain size, available from Rice Tec) and “Water Maid” (long-grain size, available from RIVIANA FOODS) were washed with tap water and soaked in tap water for one hour. Then, the rice having been soaked was drained and supplied into an IH jar rice cooker Model NJ-HS06-S (manufactured by Mitsubishi Electric Co.). Next, tap water was added so as to give a water amount 1.6 times for the former or 1.9 times for the latter as much as the uncooked rice amount. Further, TGL, TG, and GO were added thereto and dissolved, followed by cooking with the aforesaid rice cooker. Table 8 shows the amounts of the enzymes. Immediately after cooking, each cooked rice was subjected to sensory evaluation. Table 8 shows the results. The sensory evaluation was made in eight items including hardness, glutinousness, water-retention, puffiness, stickiness, non-clumping properties, elasticity, and graininess. Moreover, preference in texture balance as plain white rice was expressed in the symbols oo, o, Δ, and x. oo means “highly preferred,” o means “preferred,” Δ means “somewhat preferred,” and x means “not preferred.” The evaluation was made by four panelists.

TABLE 8
Amounts of enzymes added in cooking non-Japanese rice
varieties and sensory evaluation results thereof.

| Test lot | 1 | 2 | 3 | 4 |
|---|---|---|---|---|
| Rice used | Medium grain size | | Long grain size | |
| Water Addition ratio | 1.6 | | 1.9 | |
| TGL (U/g of uncooked rice) | — | 70.69 | — | 70.69 |
| TG (U/g of uncooked rice) | — | 0.26 | — | 0.26 |
| GO (U/g of uncooked rice) ¹ | — | 0.95 | — | 0.95 |
| GO/TGL (ratio by U) | — | 0.013 | — | 0.013 |
| GO/TG (ratio by U) | — | 3.654 | — | 3.654 |
| Sensory evaluation result | x | ∘∘ | x | ∘ |

As Table 8 shows, preferred effects as plain white rice were observed in the test lots where TGL, TG, and GO were used together, which suggests that the combined use of these enzymes can also improve the qualities of foreign rice varieties, such as medium grain size and long grain size varieties, to thereby give preferred texture.

### Example 8

150 g of uncooked rice “Hokkaido Kirara 397” was washed with tap water and soaked in tap water for one hour. After draining, the rice was supplied into an IH jar rice cooker Model NJ-HS06-S (manufactured by Mitsubishi Electric Co.). Next, tap water was added so as to give a water amount 1.5 times as much as the uncooked rice amount. Then, TGL, TG, and GO were added thereto and dissolved, followed by cooking with the rice cooker. Table 9 shows the amounts of the individual enzymes. Each cooked rice was fried in a frying pan using a seasoning blend for fried rice “Rice Cook” (manufactured by Ajinomoto Co.) for three minutes and then quickly frozen at −40° C. in a quick freezer “Blast Chiller/Shock Freezer Model QXF-012SF5” (manufactured by Fukushima Industries, Co.) to give a frozen fried rice. After storing for one week at −20° C., the frozen fried rice was microwaved and then subjected to sensory evaluation. Table 9 shows the results. The sensory evaluation was made for eight items including hardness, glutinousness, water-retention, puffiness, stickiness, non-clumping properties, elasticity, and graininess. Moreover, preference in texture balance as fried rice was expressed in the symbols oo, o, Δ, and x. oo means “highly preferred,” o means “preferred,” Δ means “somewhat preferred,” and x means “not preferred.” The evaluation was made by three panelists.

As Table 9 shows, a highly preferred effect was observed in the test lot where TGL, TG, and GO were used together, which suggests that the combined use of these enzymes enables the production of fried rice having preferred texture.

TABLE 9
Amounts of enzymes added in producing frozen fried
rice and sensory evaluation results thereof.

| Test lot | 1 | 2 |
|---|---|---|
| TGL (U/g of uncooked rice) | — | 70.69 |
| TG (U/g of uncooked rice) | — | 0.26 |
| GO (U/g of uncooked rice) ¹ | — | 0.95 |
| GO/TGL (ratio by U) | — | 0.013 |
| GO/TG (ratio by U) | — | 3.654 |
| Sensory evaluation result | x | ∘∘ |

### Example 9

150 g of uncooked rice “Hokkaido Kirara 397” was washed with tap water and soaked in tap water for one hour. After draining, the rice was supplied into an IH jar rice cooker Model NJ-HS06-S (manufactured by Mitsubishi Electric Co.). Next, tap water was added so as to give a water amount 1.5 times as much as the uncooked rice amount. Also, a seasoning mix for seasoned rice “Takikomi-tei” for rice cooked with matsutake mushroom (manufactured by Ajinomoto Co.) was added thereto. Then, TGL, TG, and GO were added thereto and dissolved, followed by cooking with the rice cooker. Table 10 shows the amounts of the individual enzymes. Each rice cooked with the seasoning mix was quickly frozen at −40° C. in a quick freezer “Blast Chiller/Shock Freezer Model QXF-012SF5” (manufactured by Fukushima Industries, Co.) to give a frozen rice cooked with the seasoning mix. After storing for one week at −20° C., the frozen rice cooked with the seasoning mix was microwaved and then subjected to sensory evaluation. Table 10 shows the results. The sensory evaluation was made for eight items including hardness, glutinousness, water-retention, puffiness, stickiness, non-clumping properties, elasticity, and graininess. Moreover, preference in texture balance as rice cooked with the seasoning mix was expressed in the symbols oo, o, Δ, and x. oo means “highly preferred,” o means “preferred,” Δ means “somewhat preferred,” and x means “not preferred.” The evaluation was made by three panelists.

TABLE 10
Amounts of enzymes added in producing frozen rice cooked with
the seasoning mix and sensory evaluation results thereof.

| Test lot | 1 | 2 |
|---|---|---|
| TGL (U/g of uncooked rice) | — | 70.69 |
| TG (U/g of uncooked rice) | — | 0.26 |
| GO (U/g of uncooked rice) ¹ | — | 0.95 |
| GO/TGL (ratio by U) | — | 0.013 |
| GO/TG (ratio by U) | — | 3.654 |
| Sensory evaluation result | x | ∘∘ |

As Table 10 shows, a highly preferred effect was observed in the test lot where TGL, TG, and GO were used together, which suggests that the combined use of these enzymes enables the production of rice cooked with the seasoning mix having preferred texture.

### INDUSTRIAL APPLICABILITY

Since the qualities of a cooked rice food can be improved according to the present invention, the invention is highly useful in the field of food industry.

Where a numerical limit or range is stated herein, the endpoints are included. Also, all values and subranges within a numerical limit or range are specifically included as if explicitly written out.

Obviously, numerous modifications and variations of the present invention are possible in light of the above teachings. It is therefore to be understood that, within the scope of the appended claims, the invention may be practiced otherwise than as specifically described herein.

All patents and other references mentioned above are incorporated in full herein by this reference, the same as if set forth at length.

1. A method for producing a cooked rice food, which comprises treating rice with:
(a) glucose oxidase; and
(b) transglutaminase and/or α-glucosidase.

2. A method according to claim 1, wherein said rice is treated with glucose oxidase and transglutaminase.

3. A method according to claim 1, wherein said rice is treated with glucose oxidase and α-glucosidase.

4. A method according to claim 1, wherein said rice is treated with glucose oxidase, transglutaminase, and α-glucosidase.

5. A method for producing a cooked rice food, which comprises treating rice with:
(a) 0.001 to 500 U of glucose oxidase, per g of uncooked rice as a raw material; and
(b) 0.0001 to 120 U of transglutaminase, per g of uncooked rice as a raw material, and/or 0.03 to 300,000 U of α-glucosidase, per g of uncooked rice as a raw material.

6. A method for producing a cooked rice food, which comprises treating rice with:
(a) 0.03 to 210 U of glucose oxidase, per g of uncooked rice as a raw material; and
(b) 0.05 to 12 U of transglutaminase, per g of uncooked rice as a raw material, and/or 15 to 150,000 U of α-glucosidase per g of uncooked rice as a raw material.

7. A method according to claim 1, wherein said rice is treated with said glucose oxidase in an amount of 0.003 to 10,000 U per U of transglutaminase and/or 0.000003 to 34 U per U of α-glucosidase.

8. A method according to claim 2, wherein said rice is treated with said glucose oxidase in an amount of 0.003 to 10,000 U per U of transglutaminase.

9. A method according to claim 3, wherein said rice is treated with said glucose oxidase in an amount of 0.000003 to 34 U per U of α-glucosidase.

10. A method according to claim 1, wherein said rice is treated with said glucose oxidase in an amount of 0.1 to 900 U per U of transglutaminase and/or 0.000006 to 3 U per U of α-glucosidase.

11. A method according to claim 2, wherein said rice is treated with said glucose oxidase in an amount of 0.1 to 900 U per U of transglutaminase.

12. A method according to claim 3, wherein said rice is treated with said glucose oxidase in an amount of 0.000006 to 3 U per U of α-glucosidase.

13. An enzyme preparation, which comprises, transglutaminase and/or α-glucosidase together with glucose oxidase.

14. An enzyme preparation according to claim 13, wherein:
said glucose oxidase and said transglutaminase are present in a ratio of 0.003 to 10,000 U of said glucose oxidase per U of said transglutaminase; and/or
said glucose oxidase and said α-glucosidase are present in a ratio of 0.000003 to 34 U of said glucose oxidase per U of said α-glucosidase.

15. An enzyme preparation according to claim 14, wherein:
said glucose oxidase and said transglutaminase are present in a ratio of 0.1 to 900 U of said glucose oxidase per U of said transglutaminase; and/or
said glucose oxidase and said α-glucosidase are present in a ratio of 0.000006 to 3 U of said glucose oxidase per U of said α-glucosidase.

16. A cooked rice food, which is prepared by a method according to claim 1.

17. A cooked rice food, which is prepared by a method according to claim 2.

18. A cooked rice food, which is prepared by a method according to claim 3.

19. A cooked rice food, which is prepared by a method according to claim 4.
